(19) United States
(12) Patent Application Publication
(10) Pub. No.: US 2024/0139465 A1
(43) Pub. Date: May 2, 2024

(54) ELECTROSTIMULATOR DEVICE FOR SLEEP INDUCTION

(71) Applicant: MUNDURAT, S.L., Anorga (Gipuzkoa) (ES)

(72) Inventor: Jon GUILLEN MONTOYA, Anorga (Gipuzkoa) (ES)

(21) Appl. No.: 18/546,867

(22) PCT Filed: Feb. 23, 2022
(86) PCT No.: PCT/ES2022/070094

§ 371 Date: Aug. 17, 2023

(30) Foreign Application Priority Data
Feb. 24, 2021 (ES) P202130154

(51) Int. Cl. A61M 21/02

(52) U.S. Cl. CPC A61M 21/02; A61M 2021/0072

(57) ABSTRACT

An electrostimulator device for inducing deep sleep having at least one bracelet-type element capable of producing impulses to be applied on the nervous system to influence the brain, wherein the bracelet-type element incorporates a functional module (1) provided with an electric pulse generator (3), there being connected to the generator (3) at least one electrode (5) arranged to transmit the electric pulses to the skin of the user's wrist in the position of use of the device for its influence on the brain.

### TECHNICAL FIELD

The present invention relates to treatments to promote deep sleep that favor regenerative processes and improve vegetative functions, proposing an electrostimulator device that achieves this function in an advantageous manner.

### STATE OF THE ART

The nerves of the body are like a “telephone connection” that transmits messages between the brain and the rest of the body, in such a way that by applying external stimuli that can be captured by the nerves, messages are sent to the brain, which reacts to adapt body conditions based on these messages.

In this sense, the wrists of the arms are areas where the nerves have many grouped fibers, which makes it possible to capture external stimuli that are applied with great effectiveness, to send information messages to the brain based on said stimuli.

Accordingly, devices are being developed to apply stimuli that make it possible to modify brain waves in favorable conditions for body relaxation and falling asleep. In this regard, the optimal conditions to be obtained are those related to deep sleep.

Deep sleep is the phase in which body functions and brain waves slow down, the phase in which cell regeneration and protein synthesis, so important for growing muscles, occurs.

Sleep consists of different phases, the phase of interest being the one that occurs in the final stages of non-REM sleep, related to deep sleep. During this phase, glucose metabolism in the brain increases, indicating that it is carrying out processes important for short- and long-term memory and learning in general. Without deep sleep, the hippocampus does not correctly store new experiences as memories, that is, it is more difficult to learn.

Also, it is during deep sleep when the pituitary gland secretes important hormones, such as human growth hormone, which lead to the growth and development of all tissues, from the skin and nails to the liver and muscles. Without deep sleep, our bodies break down from lack of maintenance. The lack of said deep sleep is also associated with diseases such as diabetes, cardiovascular diseases and heart attacks, and specifically, Alzheimer's disease.

An example of a sleep stimulation device is proposed in document U.S. Pat. No. 9,610,421, which describes a bracelet-type device for applying impulses to the wrist intended to modify the frequency of brain waves. Said document intends to stimulate physiological processes with ultra-low frequency non-vibratory tactile stimuli by applying them to the skin. These stimuli are applied at such low frequencies since these ranges are below what is commonly conceived as vibratory.

The stimulator device described in said document is of the electromechanical type, that is, that by means of an electrically actuated mechanism it produces taps on the skin, at certain frequencies, to act on the nervous system. However, due to its nature of operation and constructive embodiment, this device is limited in its effectiveness and safety of operation to obtain optimal effects in the body relaxation function that is intended through nerve stimulation, being prone to malfunctions due to its mechanical condition of action.

### OBJECT OF THE INVENTION

According to the present invention, a nerve stimulation device is proposed to induce deep sleep, the embodiment of this device being based on an electrostimulation concept that improves the functional conditions and the effectiveness of the electromechanical stimulation concept.

The device object of the invention comprises at least one bracelet-type element, provided with a strap to be attached to the user's wrist, incorporating a functional module attached to the strap that incorporates an electric current impulse generator, associated with a power supply battery and electrodes arranged to be in contact with the user's skin in the use arrangement of the device. The bracelet configuration is not limiting, being able to vary the fixing design of the device for its application in other sensitive places of the body as long as the electrodes are placed in contact with the user's skin to transmit the electric current impulses.

A device is thereby obtained which, when put into operation, generates microcurrents that give rise to low-intensity and low-frequency electric pulses, so that when the device is placed on the wrist of a user, the electric pulses act on the skin as impulses (imperceptible to the human body) that are transmitted by the nervous system to modify the frequency of brain waves. This device results in a smaller, more comfortable format that is less prone to malfunctions than known devices which, having mechanical elements for their actuation, are predisposed to malfunctions that prevent an effective result.

The electric impulses generated by the device are produced as pulse trains, generating a wave the frequency of which decreases to a minimum frequency that remains stable, for example, around 2 Hz, thereby achieving a reduction in the frequency of the brain waves that resemble the pulses generated by electrical stimulation, which favors a deep sleep, with the consequent improvement of vegetative functions. This decrease in frequency can be regulated in a more controlled manner than in known mechanical devices, so that a more efficient result is obtained as the variation in the frequency of the generated signal is more easily controllable.

However, given that each hemisphere of the brain controls the opposite half of the body, it is foreseen that the electrostimulator device comprises two bracelet-type elements, for their respective placement on the two user's wrists, both elements communicating by means of wireless transmission, to establish between them a synchronization of the production of electric pulses, with one of the elements acting as a master and the other as a slave, so that both generate totally equal electric pulse trains.

A more complete action of the influence to induce deep sleep is thereby obtained, therefore, with this embodiment, the greater the effect that is achieved for said function.

On the other hand, the functional module of each bracelet-type element of the device is provided with several electrodes to apply the electric pulses on the user's skin, for example, but without limitation, a number of six electrodes, thereby ensuring the transmission of electric pulses to the user's nervous system, avoiding the risk that if there is a callused area on the skin it could hinder the transmission of electric pulses, since with multiple electrodes it is guaranteed that at least one remains in a situation of establishing a good transmission, outside of any area that may hinder transmission.

For all these reasons, the device object of the invention has some advantageous features for inducing deep sleep, for which it is intended, being novel and preferred with respect to the conventional solutions currently known for said function.

### DESCRIPTION OF THE FIGURES

FIG. 1 shows a perspective view of an exemplary embodiment of a bracelet-type element for the electrostimulation function according to the object of the invention.

FIG. 2 is a top plan view of the bracelet-type element depicted in the previous figure.

FIG. 3 is a bottom plan view of the same bracelet-type element, with the fastening strap cut away to allow observation of the underside of the functional module.

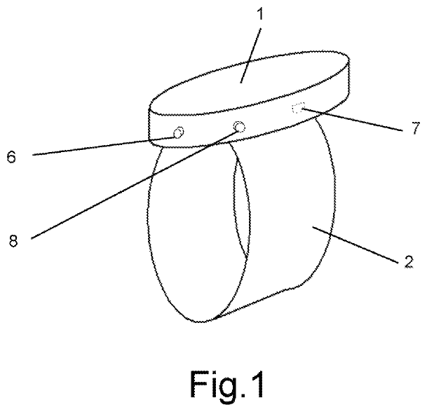

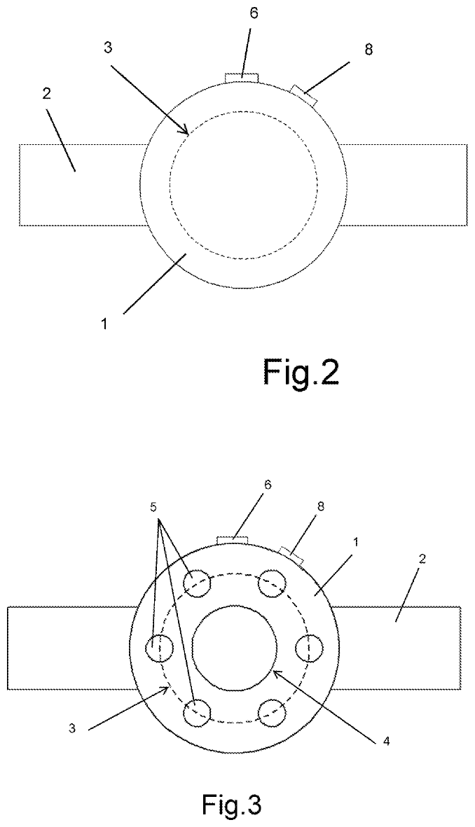

### DETAILED DESCRIPTION OF THE INVENTION

The object of the invention relates to a stimulation device to influence the brain through the nervous system, in order to achieve deep sleep.

The device comprises at least one bracelet-type element, formed by a functional module (1) attached to a strap (2) for fastening on the wrist of the user's arm, said functional assembly (1) including an electric pulse generator (3) in association with a power supply battery (4) and connected to electrodes (5) arranged to come into contact with the user's skin in the position of use of the device.

Thus, by placing the bracelet-type element on the user's wrist, when the functional module (1) is put into operation, the generator (3) produces microcurrents that give rise to low-intensity electric pulses, which are applied as impulses through electrodes (5) on the skin of the user, so that the nervous system captures the impulses and transmits them to the brain, thus obtaining a reaction whereby the brain waves adapt to the transmitted impulses.

Under these conditions, the production of electric impulses by the generator (3) is provided in the form of pulse trains, the signal being a square wave that begins at a certain frequency, for example, around 14.28 Hz, decreasing progressively up to a small stabilization frequency, for example around 2 Hz, where 2 Hz is the same frequency of brain waves in deep sleep. Therefore, due to the action of the electrostimulator device, a reduction in brain waves is achieved, “tricking the brain” and thus making it adapt that frequency as its own and enter deep sleep, with its consequent benefits and improvements of vegetative functions.

Preferably, the electric impulses are generated in a waveform with the output curve of biphasic or monophasic rectangular galvanic impulses. Preferably, the control of said impulses is carried out, at an impulse frequency between about 0.5 Hz and about 14 Hz, by a regulation of the voltage in the order of volts, for example, between 10 and 1 volts, and of current in the order of hundreds microamps, for example, from 600 uA to 100 uA.

In particular, practical examples of the applied voltages are the following:
High voltage
   Voltage (wave amplitude): 9 v DC
   Intensity: 500 uA (Max)
   Power: 4.5 mW
Medium voltage
   Voltage (wave amplitude): 6 v DC
   Intensity: 333 uA (Max)
   Power: 2 mW
Low voltage
   Voltage (wave amplitude): 3 v DC
   Intensity: 166 uA (Max)
   Power: 5 uW

Associated with the generator (3) of the functional assembly (1), it is foreseen that there is a distribution of several electrodes (5), for example, but without limitation, six electrodes (5), to make contact with the user's skin, whereby if there is a calloused part of the skin in the contact area through which the transmission of electric impulses may be difficult due to poor contact, since there are several contact electrodes (5), it is guaranteed that at least one remains outside the calloused area, thus ensuring contact for a good transmission of electric impulses without the action of the electrostimulator device losing effectiveness.

In a preferred embodiment, the electrostimulator device is made up of two bracelet-type elements, designed to be attached, respectively, to the user's two wrists, thereby transmitting electrostimulation impulses to the two hemispheres of the brain, whereby respectively each of the lateral halves of the body are related, thus obtaining a more complete and effective action of electrostimulation to adapt body conditions to deep sleep.

In this case, one of the bracelet-type elements of the electrostimulator device acts as a master and the other as a slave, establishing between them, through wireless communication, a synchronization of the electric impulses they produce, for the application of the impulses in exactly the same way on both wrists in the electrostimulation function on the user.

However, the bracelet-type element intended to act as a master can function by itself as an electrostimulator device on a user's wrist, while the bracelet-type element intended to act as a slave can only function in combination with a master bracelet-type element, since this is the one that establishes the synchronization of the operation of assembly, so that when the master bracelet-type element is put into operation, the first thing it does is a search to see if there is a respective slave bracelet-type element with which it has to communicate, to then start the electrostimulation operation on its own or in combination with the slave, as the case may be.

In any case, each bracelet-type element has a button (6) to activate and deactivate the electrical supply of the generator (3) of the respective functional assembly (1), as well as a connection socket (7) to connect a load supply of the battery (4), with the master bracelet-type element further having a button (8) to establish the start of generation of electric impulses for the electrostimulation function, the slave bracelet-type element lacking this button (8), since its start of generation of electric impulses is activated by the master bracelet-type element through wireless communication.

Alternatively, the bracelets could be used on other parts of the body, such as the ankles, since the electric impulses would affect the nervous system in the same way and could circulate through the body in the same way.

As a novel feature, this device has been found to be effective when used in animals, more specifically in horses. Therefore, by regulating the impulses at the frequencies of the nervous systems of animals, it is alternatively possible to use this device to induce deep sleep in animals, such as horses or pets such as dogs and cats.

1. An electrostimulator device for inducing deep sleep, comprising at least one bracelet-type element producing impulses to be applied on the nervous system to influence the brain, wherein the bracelet-type element comprises a functional module (1) provided with an electric pulse generator (3), there being connected to said generator (3) at least one electrode (5) arranged to transmit the electric pulses to the skin of the user's wrist in the position of use of the device for its influence on brain.

2. The electrostimulator device for inducing deep sleep according to claim 1, wherein the electric pulse generator (3) is configured to generate a wave signal the frequency of which decreases cyclically from an initially predetermined frequency to a selective frequency related to the frequency of deep sleep.

3. The electrostimulator device for inducing deep sleep according to claim 1, wherein the electric pulse generator (3) is associated with a power supply battery (4), which has a connection socket (7) for connecting an electrical load supply.

4. The electrostimulator device for inducing deep sleep according to claim 1 wherein the functional module (1) incorporates a button for activating and deactivating the connection between the generator (3) and the power supply battery (5).

5. The electrostimulator device for inducing deep sleep according to claim 1, further comprising a distribution of various electrodes (5) arc arranged in connection with the generator (3) to transmit the electric pulses.

6. The electrostimulator device for inducing deep sleep according to claim 1, further comprising two bracelet-type elements each applicable to a wrist to produce electric pulses to be applied to the nervous system for each corresponding hemisphere of the brain, both bracelet-type devices being related by means of a wireless communication that synchronizes them in the production of electric pulses, one of the bracelet-type elements acting as a master and the other as a slave of the synchronized operation.

7. The electrostimulator device for inducing deep sleep according to claim 6, wherein the bracelet-type element intended to act as a master has a button (8) to activate the start of the electric pulse production operation.
